(19) United States
(12) Patent Application Publication
(10) Pub. No.: US 2010/0312296 A1
(43) Pub. Date: Dec. 9, 2010

(54) CARDIOVERSION ELECTRODE

(76) Inventor: Richard J. GRAY, Carmel Valley, CA (US)

Correspondence Address: MCDERMOTT WILL & EMERY LLP, 18191 VON KARMAN AVE., SUITE 500, IRVINE, CA, 92612-7108, US

(21) Appl. No.: 12/645,429

(22) Filed: Dec. 22, 2009

Related U.S. Application Data
(60) Provisional application No. 61/140,577, filed on Dec. 23, 2008.

(51) Int. Cl. A61N 1/05; A61N 1/39

(52) U.S. Cl. 607/5; 607/130

(57) ABSTRACT

A device is provided for treating atrial fibrillation. A patch is inserted through a surgical incision into a patient and is configured to attach to the patient's heart. An elongate member is attached to the patch and is configured to detach from the patch after the incision is closed and the risk of postoperative atrial fibrillation has decreased. The device is configured to provide atrial defibrillation therapy to the heart upon detection of atrial fibrillation, which can include the application of electrical energy. The patch may be bioresorbable and may include bioresorbable glue for attachment to the heart. Detection of atrial fibrillation may be performed through internal or external sensors.

### RELATED APPLICATIONS

This application claims the benefit of U.S. Provisional Application Ser. No. 61/140,577, filed on Dec. 23, 2008, and titled “Cardioversion Electrode”, the entirety of which is hereby incorporated by reference.

### FIELD

The present disclosure relates to the treatment of atrial fibrillation, and in particular to defibrillating electrodes.

### BACKGROUND

Atrial fibrillation is a common cardiac arrhythmia and occurs when the orderly wavefront of activation breaks up into multiple components. Each of these activation wavefronts wanders rapidly and chaotically through the atria. This irregular activation pattern results in uncoordinated and ineffective contraction of the atria as well as a rapid and irregular ventricular rate.

Although it is not usually life threatening arrhythmia, atrial fibrillation is associated with a more than four-fold increase in risk of stroke. Approximately 15 percent of all strokes occur in people with atrial fibrillation. The strokes are caused by blood clots forming in areas of stagnant blood flow as a result of prolonged atrial fibrillation. In addition, patients afflicted with atrial fibrillation generally experience palpitations of the heart and may even experience dizziness.

### SUMMARY

Atrial fibrillation is common after open heart surgical procedures and has a peak incidence of occurrence between 5 to 7 days postoperation. Disclosed herein are methods, devices, and systems that are configured to treat postoperative atrial fibrillation. In some embodiments, a device, having both bioresorbable materials and nonbioresorbable materials, is implanted in the patient's body. After a period of time that is associated with a reduced risk of postoperative atrial fibrillation, the nonbioresorbable materials are non-surgically removed from the body, and the bioresorbable materials are resorbed by the body.

In some embodiments, an incision is made in the patient's skin. A patch, having an elongate member attached thereto, is advanced through the incision and to a point adjacent the atrial region of the heart. The patch is attached to a portion of the atria of the patient's heart with the elongate member extending from the patch, through the incision, to a location outside the patient's body. The incision is then closed around the elongate member. During the postoperative period, the heart is monitored for signs of atrial fibrillation. If atrial fibrillation is detected, atrial defibrillation therapy can be applied, which can include, for example, application of electric energy through the elongate member. In some embodiments, the elongate member provides a low density charge to the atria of the patient's heart by providing a configuration that increases the area of electrical conductivity between the elongate member and the atria. In some embodiments, the patch itself can be electrically conductive and facilitate distribution of the electric energy.

After a monitoring period, which corresponds to a postoperative time period for a reduced risk of postoperative atrial fibrillation, the elongate member can be removed from the patient by pulling, from outside the patient, the elongate member through the closed incision. As the elongate member is pulled from outside the patient, the elongate member is decoupled from the patch, and is withdrawn from the patient through the closed incision. The remaining portion of the patch is resorbed by the body.

This disclosure includes a medical device for treating temporary atrial fibrillation comprising a patch configured to attach to an atrial region of the heart, and an elongate member attached to the patch, where the elongate member is configured to be detachable from the heart after the patch has been configured to attach to the atrial region of the heart. In some embodiments, the device is configured to apply atrial defibrillation therapy to the heart upon detection of atrial fibrillation.

In accordance with some embodiments, the patch may be bioresorbable, and may be configured to attach to the atrial region of the heart utilizing bioresorbable glue. The atrial defibrillation therapy can include application of electrical energy, wherein the electrical energy is up to about 150 joules. In some embodiments, the elongate member is an epicardial electrode, where the epicardial electrode is configured to provide low charge density and is configured in a coil, rectangular, triangular, polygonal, circular, star, oval, or squiggly line shape. According to certain embodiments, detection of atrial fibrillation is performed through internal sensors.

Also described is a method for treating temporary atrial fibrillation, the method comprising the steps of disposing a device in an atrial region of the heart, wherein the device comprises a patch and an elongate member attached to the patch, applying atrial defibrillation therapy to the heart upon detection of atrial fibrillation, and detaching the elongate member from the heart after the device is disposed in the atrial region of the heart. According to some embodiments, the method for treating temporary atrial fibrillation comprises the step of detecting atrial fibrillation.

Described herein is a system for treating temporary atrial fibrillation comprising an internal control and an external control operatively connected to the internal control. In accordance with some embodiments of the present disclosure, the internal control further comprises a patch configured to attach to an atrial region of the heart, and an elongate member attached to the patch, where the elongate member is configured to be detachable from the heart after the patch has been configured to attach to the atrial region of the heart. In accordance with additional embodiments of the present disclosure, the external control comprises a detector configured to detect atrial fibrillation. A processor can also be attached to the detector, and the processor can be configured to instruct the internal control to apply atrial defibrillation therapy to the heart upon detection of atrial fibrillation.

In some embodiments, described herein is a device, for treating postoperative atrial fibrillation, comprising a patch configured to attach to an atrial region of a patient's heart, the patch having a substrate layer and an adhesion layer; and an elongate member coupled to the patch, the elongate member extending through at least one of the substrate layer and the adhesion layer and being configured to extend from outside the patient, through an incision in the patient, and to the patch. In certain embodiments, after the patch is attached to the atrial region of the heart, the elongate member is configured to apply atrial defibrillation therapy to the heart upon detection of atrial fibrillation, and the elongate member is configured to be decouple from the patch after the incision in the patient is closed and after a monitoring period.

In some embodiments, the patch is bioresorbable, and may be configured to attach to the atrial region of the heart with a bioresorbable glue. In certain embodiments, the bioresorbable glue is conductive of electricity. In some embodiments, the atrial defibrillation therapy comprises application of electrical energy, which can be up to about 150 joules. In certain embodiments, the elongate member comprises an epicardial electrode, and the epicardial electrode can be configured, for example, in a coil, rectangular, triangular, polygonal, circular, star, oval, accordion, or irregular line shape. In some embodiments, detection of atrial fibrillation is performed through sensors internal to the patient. In certain embodiments, the patch is configured to remain attached to the atrial region of the heart during the monitoring period, which comprises a time period for a risk of atrial fibrillation to decrease at least more than 50% of an initial risk of atrial fibrillation following implantation of the device. In some embodiments, the adhesion layer of the patch is configured to attach the device to the atrial region of the heart with an adhesive force that is greater than the coupling between the patch and the elongate member.

Also described herein is a method, of treating atrial fibrillation, comprising creating a surgical opening in a patient; advancing a patch, having an adhesive layer and coupled to an elongate member, through the surgical opening and to a position adjacent the patient's heart; attaching the patch to an atrial region of the heart, the elongate member extending from outside the patient, through the surgical opening, and coupled to the adhesive layer of the patch at the atrial region of the heart; closing substantially the surgical opening with the elongate member extending from outside the body, through the substantially closed surgical opening, to the patch attached to the atrial region of the heart; applying atrial defibrillation therapy to the heart through the elongate member if atrial fibrillation is detected; and decoupling the elongate member from the adhesive layer after the surgical opening is substantially closed.

In some embodiments, the method further comprises removing the elongate member from the patient through the substantially closed surgical opening. In certain embodiments, closing substantially the surgical opening comprises closing the surgical opening around the elongate member. In some embodiments, attaching the patch comprises attaching the patch to the atrial region with bioresorbable glue. The method can further include conducting electricity through the bioresorbable glue. In some embodiments, applying atrial defibrillation therapy comprises applying electrical energy, which can be up to about 150 joules of electrical energy. The method can also include detecting atrial fibrillation.

In some embodiments, a system, for treating atrial fibrillation, is described, comprising a patch configured to attach to an atrial region of a patient's heart, the patch being sized and configured to be inserted into the patient through an open incision in the patient's skin; an elongate member having a distal portion coupled to the patch and a proximal portion configured to extend outside the patient through the open incision in the patient's skin when the patch is attached to the atrial region of the heart, the distal portion being configured to decoupled from the patch after the incision in the patient's skin is closed; a detector configured to detect atrial fibrillation of the patient's heart; and a processor, in communication with the detector, configured to direct the application of atrial defibrillation therapy through the elongate member upon detection of atrial fibrillation by the detector.

In some embodiments, the patch is bioresorbable, and the patch can include a bioresorbable glue. In certain embodiments, the atrial defibrillation therapy comprises the application of electrical energy, which can be up to about 150 joules. In some embodiments, the elongate member comprises an epicardial electrode, and in certain embodiments, the distal portion of the elongate member is configured in, for example, a coil, rectangular, triangular, polygonal, circular, star, oval, or squiggly line shape. In some embodiments, the detector is configured to be implanted inside the patient.

Also described herein is a device, for treating postoperative atrial fibrillation, comprising: an attachment member configured to attach to an atrial region of a patient's heart, the attachment member having an adhesion portion; and an elongate member coupled to the attachment member and configured to extend from outside the patient, through the patient's skin, and to the attachment member when the adhesion portion is attached to the atrial region of the heart. In some embodiments, the elongate member is configured to deliver electrical energy to the heart, sufficient to treat atrial fibrillation, upon detection of atrial fibrillation after the attachment member is attached to the atrial region of the heart. In some embodiments the elongate member is configured such that, after the attachment member is attached to the atrial region, the elongate member is decouplable from the attachment member and is removable from the atrial region by withdrawing the elongate member through the patient's skin.

In some embodiments, the elongate member extends into the adhesion portion of the attachment member. In some embodiments, the attachment member is bioresorbable. In certain embodiments, the attachment member is configured to attach to the atrial region of the heart with a bioresorbable glue. The bioresorbable glue can be conductive of electricity according to certain embodiments. In some embodiments, the electrical energy is up to about 150 joules. In some embodiments, the elongate member comprises an epicardial electrode. In some embodiments, the epicardial electrode is configured in a coil, rectangular, triangular, polygonal, circular, star, oval, accordion, or irregular line shape. In some embodiments, detection of atrial fibrillation is performed through sensors internal to the patient. In certain embodiments, the attachment member is configured to remain attached to the atrial region of the heart during a monitoring period, which comprises a time period for a risk of atrial fibrillation to decrease at least more than 50% from an initial risk of atrial fibrillation following implantation of the device. In some embodiments, the adhesion portion of the attachment member is configured to attach the device to the atrial region of the heart with an adhesive force that is greater than the coupling between the attachment member and the elongate member.

Also described herein is a method, of treating atrial fibrillation, comprising creating an opening in a patient's skin; advancing an attachment member, having an adhesive layer coupled with an elongate member, through the opening and to a position adjacent the patient's heart; attaching the attachment member to an atrial region of the heart, the elongate member extending from outside the patient, through the patient's skin, and coupled to the adhesive layer of the attachment member at the atrial region of the heart; at least partially closing the opening, with the elongate member extending from outside the body, through the patient's skin, to the attachment member attached to the atrial region of the heart; and decoupling the elongate member from the adhesive layer, and removing the elongate body from the patient, after the opening is substantially closed.

In some embodiments, the method further comprises applying atrial defibrillation therapy to the heart through the elongate member to treat atrial fibrillation. In some embodiments, the method further comprises removing the elongate member from the patient without reopening the opening. In some embodiments, at least partially closing the opening comprises closing the opening around the elongate member. In certain embodiments, attaching the attachment member comprises attaching the attachment member to the atrial region with bioresorbable glue. In some embodiments, the method further comprises conducting electricity through the bioresorbable glue. In some embodiments, applying atrial defibrillation therapy comprises applying electrical energy. In some embodiments, applying electrical energy comprises applying up to about 150 joules of electrical energy. In some embodiments, the method further comprises detecting atrial fibrillation.

Also described herein is a system, for treating atrial fibrillation, comprising: an attachment member configured to attach to an atrial region of a patient's heart, the attachment member being sized and configured to be inserted into the patient through an open incision in the patient's skin; an elongate member having a distal portion coupled to the attachment member and a proximal portion configured to extend outside the patient through the patient's skin when the attachment member is attached to the atrial region of the heart, the distal portion being configured to be decoupled from the attachment member and is removable from the atrial region by withdrawing the elongate member through the patient's skin after the incision in the patient's skin is closed; a detector configured to detect atrial fibrillation of the patient's heart; and a processor, in communication with the detector, configured to direct the application of atrial defibrillation therapy through the elongate member upon detection of atrial fibrillation by the detector.

In some embodiments, the attachment member is bioresorbable. In some embodiments, the attachment member comprises bioresorbable glue. In some embodiments, the atrial defibrillation therapy comprises the application of electrical energy. In some embodiments, the electrical energy is up to about 150 joules. In some embodiments, the elongate member comprises an epicardial electrode. In some embodiments, the distal portion of the elongate member is configured in a coil, rectangular, triangular, polygonal, circular, star, oval, or irregular line shape. In some embodiments, the detector is configured to be implanted inside the patient.

Other features and advantages are inherent in the device and disclosed or will become apparent to those skilled in the art from the following detailed description and its accompanying drawings.

### BRIEF DESCRIPTION OF THE DRAWINGS

The accompanying drawings, which are included to provide further understanding of the disclosure and are incorporated in and constitute a part of this specification, illustrate embodiments of the disclosure and, together with the description, serve to explain the principles of the disclosure. In the drawings:

FIG. 1 illustrates embodiments of the device as applied to the heart.

FIGS. 2A, 2B, 2C, 2D, 2E, 2F, and 2G illustrate embodiments of a cross sectional view of the device.

FIGS. 3A, 3B, 3C, 3D, 3E, 3F, 3G, 3H, and 3I illustrate various embodiments of the device.

FIG. 4 schematically illustrates embodiments of the device placed in relation to the patient.

FIGS. 5A, 5B, 5C, 5D, 5E, 5F, and 5G illustrate embodiments of the device.

FIGS. 6A, 6B, 6C, and 6D are schematic illustrations of implanting and removing the device.

FIG. 7 illustrates embodiments of the device with a control system.

FIG. 8 illustrates embodiments of an external configuration of the control system of the device.

FIG. 9 illustrates a flowchart of a method for treating atrial fibrillation.

FIG. 10 illustrates a method for treating temporary atrial fibrillation, in accordance with various embodiments of the disclosure.

FIG. 11 illustrates a method of treating atrial fibrillation, in accordance with various embodiments of the disclosure.

FIG. 12 illustrates a method of treating atrial fibrillation, in accordance with various embodiments of the disclosure.

### DETAILED DESCRIPTION

In the following description, reference is made to the accompanying drawings that form a part hereof, and in which is shown by way of illustration specific embodiments of this disclosure. It is to be understood that other embodiments may be utilized and structural changes may be made without departing from the scope of the present disclosure.

Postoperative atrial fibrillation after cardiac surgery is the most common and significant complication of more than 1 million annual cardiothoracic surgery procedures, such as coronary bypass grafts and aortic heart valve repairs. Postoperative atrial fibrillation occurs in 20-40% of coronary bypass and 60% of heart valve repair patients. The rate of atrial fibrillation after cardiac surgery in the 1970s was about 10%, and is now consistently at least 30%, and much higher in older patients or those undergoing valve surgery. Atrial fibrillation occurring after open heart surgical procedures has a peak incidence of occurrence between 3 to 7 days after surgery. Its occurrence is associated with worse morbidity and longer intensive-care hospitalization. To illustrate, according to various studies, the hospitalization time of bypass patients who experience atrial fibrillation is increased by an average of 2 to 4 days that are not reimbursed to the hospital. The economic impact of this prolongation exceeds $900 million per year in the United States alone. Aside from the economic impact, development of atrial fibrillation immediately after coronary artery bypass surgery results in a significantly higher (around two to three fold) risk of postoperative stroke. Postoperative atrial fibrillation has also been shown to independently predict postoperative delirium and neurocognitive decline.

Postoperative atrial fibrillation is a vexing problem and a major unresolved complication of cardiothoracic surgeries. The onset of clinically relevant atrial fibrillation cannot be predicted in individual patients; advanced age is the only generally accepted predictor.

In one approach, atrial fibrillation can be corrected by a discharge of electrical energy to the heart through the skin of the patient by way of an external defibrillator. This treatment is commonly referred to as synchronized cardioversion and involves applying electrical defibrillating energy to the heart in synchronism with a detected ventricular electrical activation (R wave) of the heart. However, this treatment can be very painful and inconvenient. As used herein, the term “atrial defibrillation” is a broad term and is intended to have its ordinary meaning, which includes, without limitation, electrical or chemical cardioversion and other treatments for atrial fibrillation that convert the arrhythmia to sinus rhythm or other acceptable cardiac rhythm.

Drugs are available for reducing the incidence of, or treating, atrial fibrillation. However, these drugs have many side effects and many patients are resistant to them which can greatly reduce their therapeutic effect. For example, the Food and Drug Administration approved ibutilide as a treatment for atrial fibrillation that can be administered intravenously. This option is attractive because it may obviate the need for sedation or general anesthesia and a powerful shock. However, ibutilide is effective in less than about one third of patients with atrial fibrillation. Many drugs used to prevent atrial fibrillation have systemic and cardiac toxicities when given in the traditional manner to achieve sufficient cardiac concentration to be effective. In many cases, drugs can effectively be used in conjunction with other treatments.

Patients with persistent or recurrent atrial fibrillation require chronic therapy of their arrhythmia. Coumadin, an anticoagulant, is usually given to these patients to reduce the risk of stroke. Antiarrhythmic drugs may decrease the frequency and severity of atrial fibrillation episodes, but can be associated with significant side effects, including the possibility of proarrhythmia. When the risks and toxicity of antiarrhythmic drugs outweigh their potential benefit, patients are left in atrial fibrillation and palliated with medications that slow the heart rate. Moreover, relatively healthy patients who undergo open heart surgery may not need these types of drugs for long term treatment of atrial fibrillation. Thus, what is needed is a relatively convenient and simple treatment for temporary atrial fibrillation occurring post operation without the need for drugs.

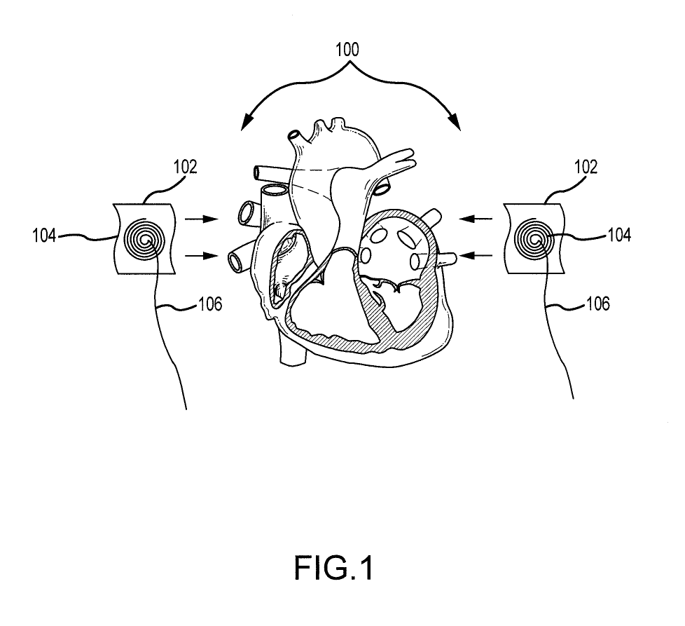

FIG. 1 illustrates the device as applied to the heart, in accordance with some embodiments.

In accordance with some embodiments of the present disclosure, device 100 may be disposed in the heart. For example, patch 102, which is also described herein as an attachment member, may be configured to attach to the atrial or ventricular regions of the heart. Additionally, patch 102 may also be attached to the veins or arteries. Patch 102 may be attached by using fastener 104, which may be adhesives, glue, needles, screws, tape, magnets, suture, or combinations of these materials. For example, fastener 104 can be used as an adhesive to allow patch 102 to stick to the heart. In other embodiments of the present disclosure, fastener 104 can be a suture where patch 102 is sewn onto the heart. Fastener 104 may also act as a needle to allow the insertion of patch 102 into the inner regions of the heart.

FIG. 1 shows two devices disposed on the heart. However, the present disclosure may involve just one device disposed in the heart or as many as needed for different types of treatment. Without departing from the scope of the present disclosure, device 100 is capable of treating various types of cardiac arrhythmia to bring the heart back to sinus rhythm. For example, device 100 can be used for treating atrial and ventricular fibrillation, premature atrial contractions, multifocal atrial tachycardia, atrial flutter, supraventricular tachycardia, AV nodal reentrant tachycardia, junctional rhythm, tachycardia, Wolff-Parkinson-White syndrome, premature ventricular contraction, accelerated idioventricular rhythm, or ventricular tachycardia.

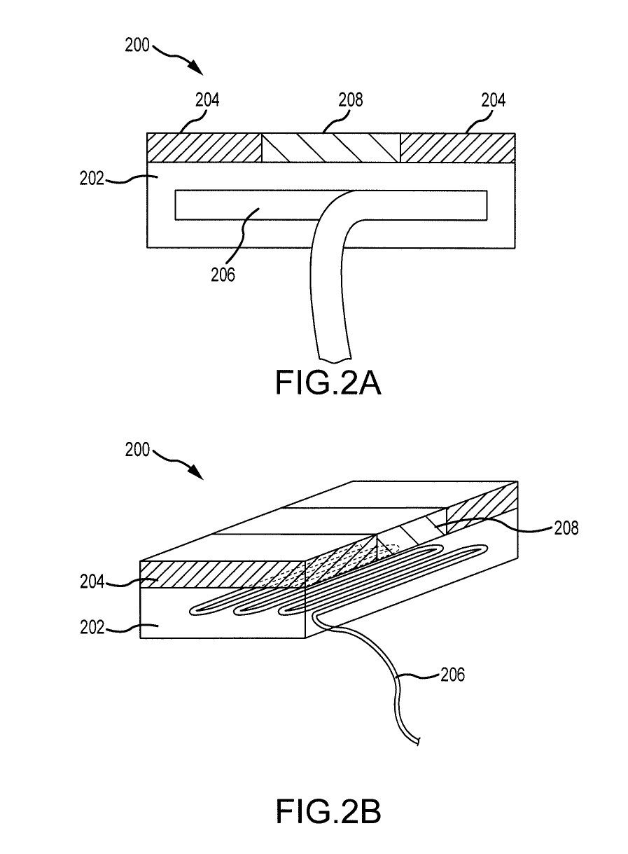

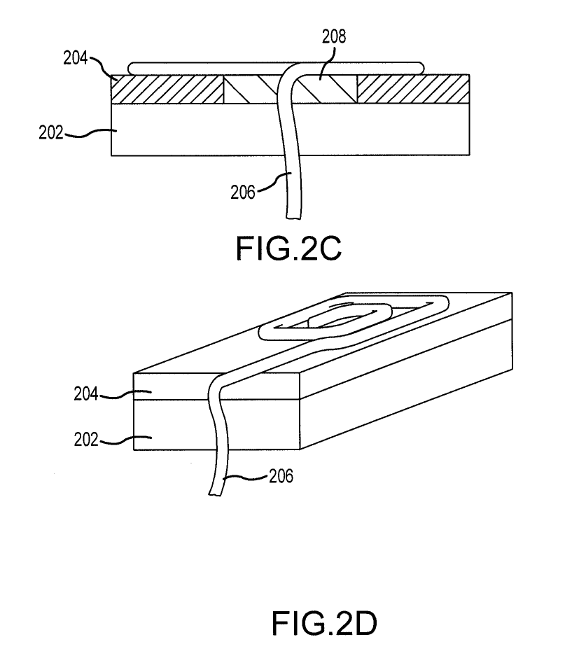

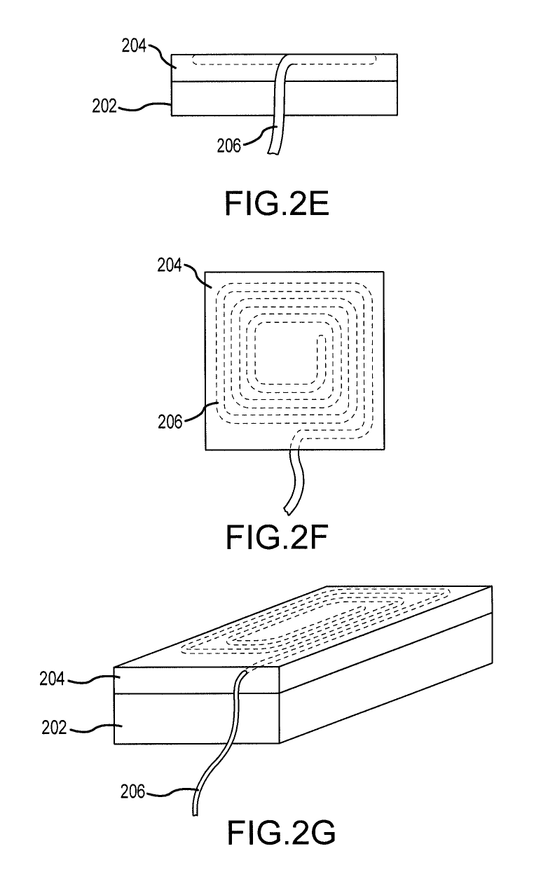

FIG. 2A illustrates a cross sectional, close up view of the device, in accordance with some embodiments of the present disclosure, and FIG. 2B illustrates a perspective view of embodiments of the device.

According to embodiments of the present disclosure, line 206 is attached to patch 202. Line 206 may be a wire, suture, electrode, catheter, string, cord, thread, rope, tube, braid or any combinations of these materials. Line 206 may be encapsulated by patch 202, lie on top or below patch 202, or wrap around patch 202. In accordance with some embodiments of the present disclosure, line 206 may be a pacing electrode that may be unipolar, bipolar, epicardial, or endocardial. Device 200 may be configured to apply atrial defibrillation therapy to the heart upon detection of atrial fibrillation, which may include applying electrical energy to the heart, application of drug therapy, and combinations thereof in accordance with embodiments of the present disclosure. For example, the amount of electrical energy may be up to about 150 joules.

In some embodiments, the amount of electrical energy may range between about 130 joules and about 170 joules, and in some embodiments, the energy may range between about 100 joules and about 200 joules. In some embodiments, the energy may be less than about 100 joules and greater than about 200 joules.

In accordance with some embodiments of the present disclosure, patch 202 may be made of linen, cotton, nylon, paper, plastic, metal, wood, or any combinations of these materials. Without departing from the scope of the present disclosure, patch 202 may also be bioresorbable or biodegradable. For example, patch 202 may be made of bioresorbable or biodegradable polymers such as PLA, PLLA, PDLLA, PGA, PGA-TMC, PGLA, LPLA, DLPLA, PCL, PDO, DLPLG, or combinations of these materials. Patch 202 is not limited to a rectangular shape, but may be other shapes as well such as ovular, polygonal, cylindrical, or any other combinations of these or other shapes. In some embodiments, patch 202 is configured not to be substantially conductive of electricity. For example, if the line 206 is on one side of the patch 202, the exposed side of the line 206 can be used for electrical atrial defibrillation therapy, and the patch 202 can insulate adjacent body tissue from the electrical treatment.

Patch 202 may be configured to attach to the heart utilizing fastener 204. Fastener 204 may be adhesives, glue, needles, screws, tape, magnets, or suture, or combinations of these materials. In some embodiments of the present disclosure, fastener 204 may also be bioresorbable or biodegradable. For example, fastener 204 may be bioresorbable glue such as gelatin, resorcinol and formaldehyde; polypeptide isolated from the byssus of the mussel; glue based on fibrinogen and thrombin (e.g., Tissucol, Beriplast, or Biocol); crosslinked natural collagen; butyl-2-cyanoacrylate glue; or combinations of these materials or other materials.

In some embodiments, fastener 204 is substantially conductive to electrical energy. This can facilitate in low charge density of electrical atrial defibrillation therapy. In such embodiments, the electrical charge can be supplied to the device 200 by line 206, and once the charge reaches the patch 202, the electrical charge can be applied through the line 206 and through the fastener 204 to spread the effective treatment area of the charge. In some embodiments, fastener 204 is not substantially conductive to electrical energy, and low charge density is achieved through the configuration of the line 206 within, or on, the patch 202.

Although shown separately, fastener 204 may be combined into patch 202 to give patch 202 fastening qualities. Furthermore, in accordance with some embodiments of the present disclosure, additive 208 may be added to patch 202 to provide extra functioning such as anticoagulation, atrial defibrillation, ventricular defibrillation, or other antiarrythmic treatment. Thus, additive 208 may include different drugs for different treatments. For example, to control heart rate, additive 208 may include Digoxin (Lanoxin), Diltiazem (Cardizem), Verapamil (Calan, Isoptin), Esmolol (Brevibloc), Metoprolol (Lopressor), Propranolol (Inderal), or combinations of these materials or other materials. For antiarrhythmic therapies, additive 208 may include Procainamide (Procainamide Hydrochloride Injection), Quinidine (Quinaglute), Propafenone (Rythmol), Flecainide (Tambocor), Amiodarone (Cordarone), Sotalol (Betapace), Ibutilide (Corvert), or combinations of these materials or other materials.

In accordance with some embodiments of the present disclosure, once device 200 is disposed on heart tissue, additive 208 may be applied directly to the heart without the need for intravenous or oral application of drugs. Additive 208 may be placed above or below patch 202, or may be combined with patch 202. Additive 208 may also be encapsulated by patch 202. Thus, the rate at which additive 208 may be applied to the heart can be controlled through its placement relative to patch 202. For example, if the delayed application of additive 208 is desired, additive 208 may be placed behind a bioresorbable patch 202. Thus, additive 208 may be applied only after patch 202 has been fully resorbed by the body.

FIGS. 2C and 2D illustrates some embodiments in which the line 206 extends along a surface of the patch 202. In these figures, the line 206 is depicted as extending on one side of the fastener 204 and the additive 208. In some embodiments, the fastener 204 and/or additive 208 can be pliable in comparison to the patch 202 and/or line 206. For example, when applying the patch 202, depicted in FIGS. 2C and 2D, to heart tissue, the patch 202 can be pressed against the heart tissue, and the fastener 204 can mold around the line 206 to contact the heart tissue.

In some embodiments, such as those depicted in FIGS. 2E, 2F, and 2G, the line 206 can be positioned at or just below the surface of the fastener 204 of the patch 202. In these embodiments, as with those depicted elsewhere herein, the fastener 204 can be pliable such that application of force when attaching the patch 202 to the heart tissue will cause molding of the patch 202 around the heart tissue and allow close deployment of the line 206 to the heart tissue. In some embodiments, the adhesive 204 is not so pliable, and merely keeps the patch 202 adhered to the heart tissue during a period that the heart is monitored.

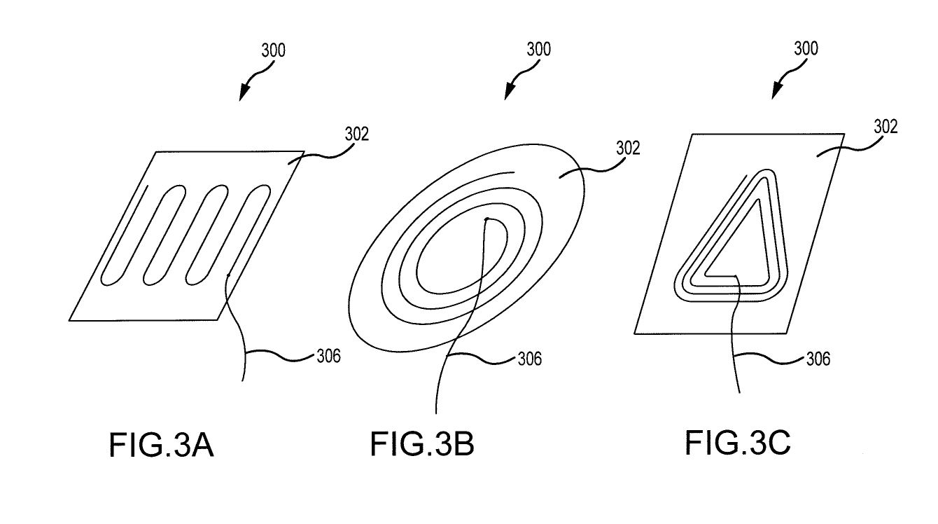

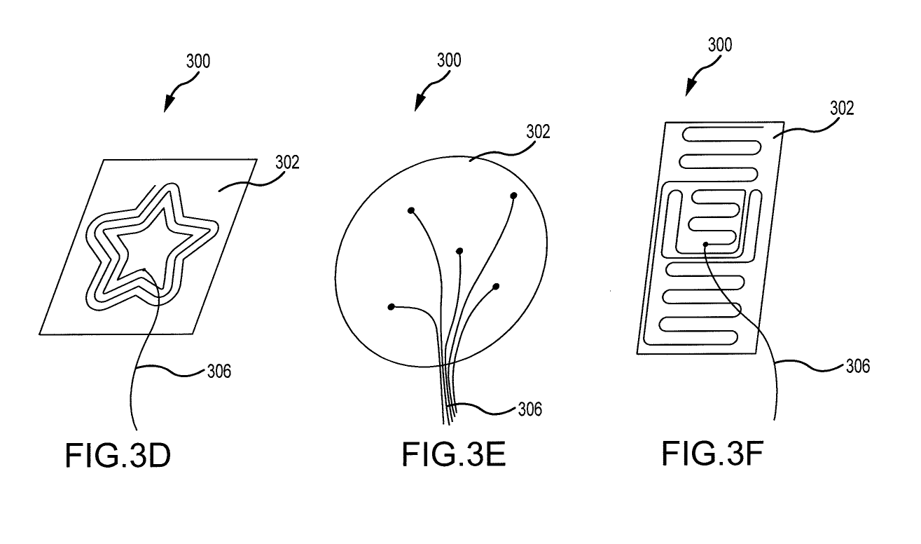

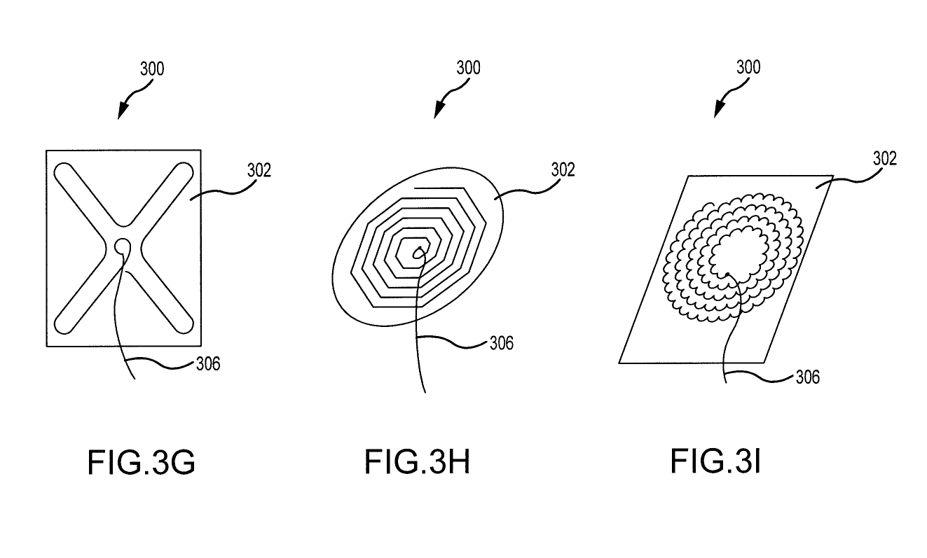

FIGS. 3A, 3B, 3C, 3D, 3E, 3F, 3G, 3H, and 3I illustrate various configurations of the device, in accordance with several embodiments of the present disclosure.

Device 300 may be configured to apply atrial defibrillation therapy, which can include electrical energy, to the heart upon detection of atrial fibrillation. Line 306 may be a pacing electrode that may be unipolar, bipolar, epicardial, or endocardial. Line 306 may be configured to provide low charge density of electrical energy to the heart. Low charge density provides a charge of electrical energy applied over a large area of tissue rather than applying electrical energy at a single point, which could create a high charge density of electrical energy and could damage or harm the tissue at that single point. Additionally, high charge density of electrical energy may not achieve the desired defibrillation therapy. For example, in accordance with some embodiments of the present disclosure, Line 306 may be wound up in a swirl, circular, rectangular, triangular, polygonal, squiggly-shape, irregular configuration, or combinations of these configurations or other configurations, without departing from the present scope of the disclosure.

Configuration of the line 306 within the patch 302 can be modified to facilitate both application of the atrial defibrillation therapy and the removal of the device 300. For example, is some embodiments, the device 300 is implanted through an incision created during a surgical procedure. After the procedure is completed, and the incision is closed, the line 306 may remain outside the body of the patient until a time that the risk for atrial fibrillation has subsided. In some embodiments, this reduced risk level may be when the risk of atrial fibrillation is 20% lower than immediately following the surgical procedure. In some embodiments, the risk level may be 30%, 40%, 50%, 60%, 75%, or 90%. In some embodiments, the patch 302 may remain within the body, attached to the heart tissue, having the line 306 extending from the patch 302 outside the body through the closed incision until the risk of atrial fibrillation has decreased 90%, 95%, 98%, or 99% from the time immediately following the surgical procedure. The atrial fibrillation risk levels can be determined, for example, through clinical trials that determine how often atrial fibrillation occurs following the surgical procedure performed.

Upon reaching the time that the risk level has decreased to a determined level, line 306 may be detached from either patch 302 or the heart by pulling out line 306 from outside the patient's body through the closed incision. This will allow for nonsurgical removal of the line 306 from the patient's body. The device 300 can be configured such that the resorption time of the patch 302 substantially coincides with the time of attaining the decreased risk level. As the line 306 is withdrawn from the patient's body, for example, line 306 may unravel from either patch 302 or the heart, which would allow line 306 to be easily removed from the patient's body through a small orifice without the need for invasive surgery. In some embodiments of the present disclosure, line 306 may be configured in such a way that when line 306 is detached, it will unwind and separate from patch 302, leaving patch 302 attached to the heart. In some embodiments of the present disclosure, patch 302 may be fully or partially resorbed into the patient's body, and line 306 will be able to detach from either patch 302 or the heart. In certain embodiments, line 306 may be configured so that once it is detached, patch 302 may also be detached from the heart to remove device 300 from the patient's body. Accordingly, provided herein are embodiments of devices that provide for the surgical implantation in the body of a patient of an atrial defibrillation therapy device and the nonsurgical removal of the device from the patient's body.

Line 306 may include more than one electrode. For example, line 306 may split into several electrodes attached to patch 302 at different points. As such, the charge density may be lower because electrical energy may be applied at lower levels to points that are spread out over patch 302. In this example, line 306 may also be detached from either patch 302 or the heart when line 306 is pulled from outside the patient's body. The split electrodes of line 306 may then combine to form substantially one single line for easier removal from the patient's body.

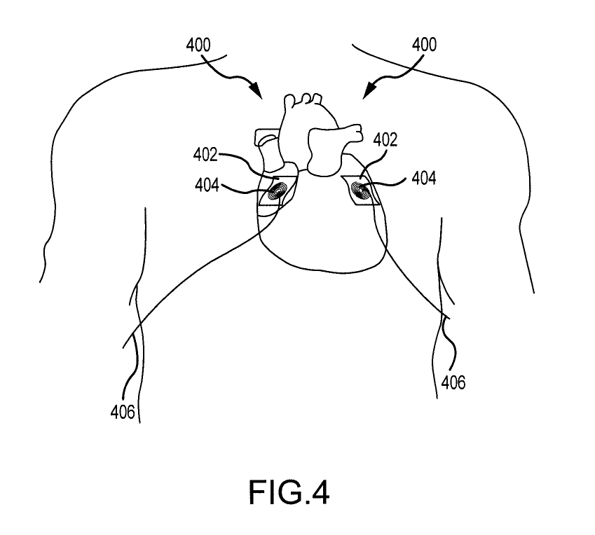

FIG. 4 illustrates the device 400 placed in relation to the patient, and in particular in relation to the patient's heart, in accordance with some embodiments of the present disclosure.

After open heart surgery, a patient is confronted with the threat of atrial fibrillation for several days. In accordance with some embodiments of the present disclosure, device 400 may be disposed in or on the patient's heart at the conclusion of the surgery. The implantation of device 400 will be relatively easy since the patient's chest will already be open from the surgery. In other applications, the device 400 can be applied laparoscopically. Device 400 may be attached to the atrial regions of the heart utilizing fastener 404. Fastener 404 may be adhesives, glue, needles, screws, tape, magnets, or suture, or combinations of these materials or other materials. Device 400 may be disposed in other regions of the heart such as the ventricular regions, and in some embodiments, the device 400 can be modified in size, shape, and configuration of the line 406 to perform ventricular therapy (e.g., ventricular pacing).

In application, a patient's chest may be closed up from open heart surgery, leaving a portion of device 400 outside of the patient's body and extending through the substantially closed opening through which surgery was performed. For example, line 406 may be attached to patch 402 in such a way that at least a portion of line 406 is still outside the patient's body at the conclusion of surgery, leaving open a small orifice in the patient's chest so that line 406 may extend from inside the body to the outside. To ensure that postoperative atrial fibrillation will be treated, a portion of device 400 may be left inside the patient for several days after surgery.

In some embodiments, device 400 may be left inside the patient for 5-7 days after surgery. In some embodiments, device 400 may remain within the patient for 3-6 days or for 6-9 days. In some embodiments, device 400 may remain within the patient for less than 3 days or more than 9 days. In some embodiments, device 400 may be left in the patient for either a shorter time or a longer time depending on the type of monitoring or treatment that is needed. The monitoring period, or the time required for the risk of fibrillation to reach a determined level, can be different for each patient depending on the patient's age, weight, history, surgical treatment, and other factors that might influence the risk of postoperative atrial fibrillation.

In some applications, the heart is monitored for various activity after device 400 has been disposed in the heart. For example, during this time, the heart may be monitored for heart rate, sinus rhythm, atrial fibrillation, or ventricular fibrillation. In accordance with some embodiments of the present disclosure, device 400 may apply atrial defibrillation therapy, which may include applying electrical energy to the heart and/or other types of drug therapy, and in some embodiments of the present disclosure, device 400 may apply other therapies for the treatment of different conditions without departing from the scope of the present disclosure. For example, device 400 may be configured to apply ventricular defibrillation therapy to the heart upon detection of ventricular fibrillation. Device 400 may also be configured to apply anticoagulation therapy to the heart to prevent or reduce the formation of blood clots. This may be useful to prevent or reduce the risk of stroke caused by the build up of clotted blood gathered in the chambers of the heart.

In some embodiments, patch 402, fastener 404, and/or line 406 may be removed by being resorbed by the patient's body. Different bioresorbable or biodegradable materials may be used to set the amount of time it would take for either patch 402, fastener 404, or line 406 to be resorbed. For example, patch 402 and fastener 404 may be configured so that they would be resorbed into the body more than 7 days after a patient's open heart surgery. In some embodiments, the materials for the patch 402 are configured to be resorbed by the body after the time provided above for a determined reduced risk level of fibrillation to be attained. In some embodiments, the material is configured to soften at about the same time are the determined reduced risk level to facilitate decoupling of the line 406 with the patch 402. In some embodiments, the coupling between the patch 402 and the line 406 is configured to be less than the attachment between the fastener 404 and the heart tissue. In such embodiments, withdrawal of the line 406 from outside the body will first decoupled the line 406 from the patch 402 before withdrawing the line 406 outside the body.

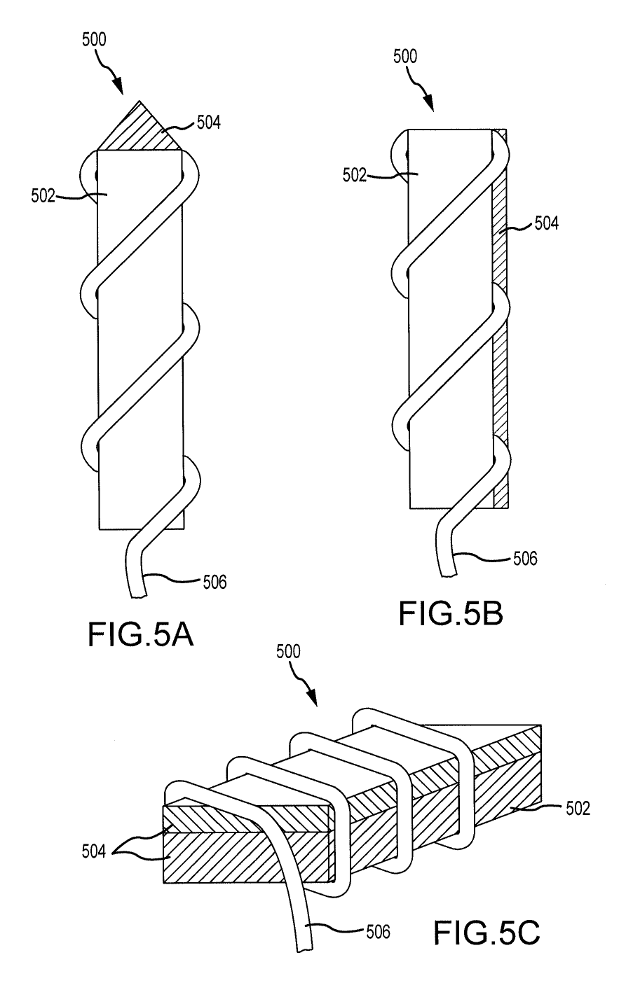

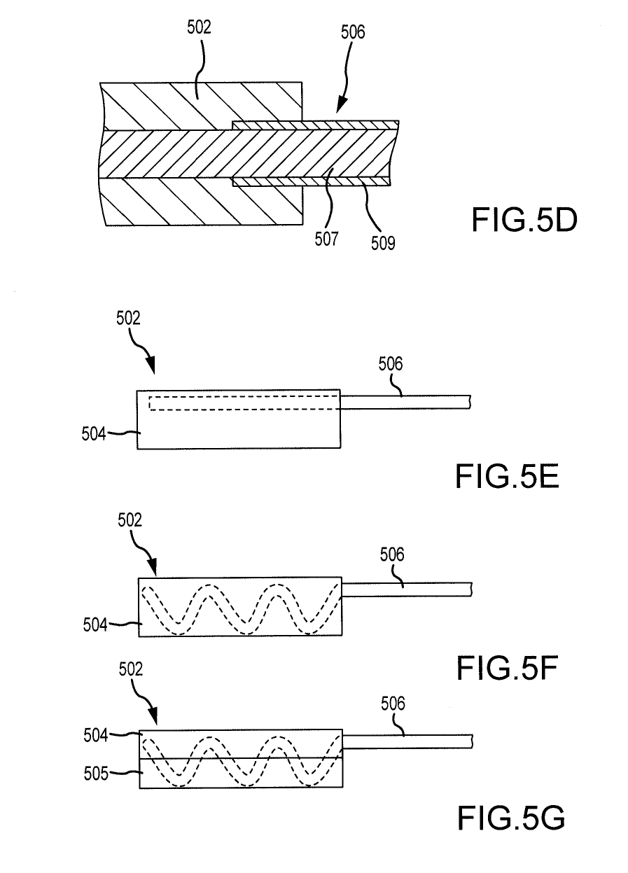

FIGS. 5A, 5B, 5C, 5D, 5E, 5F, and 5G illustrate views of embodiments of the device, in accordance with some embodiments of the present disclosure. As shown in FIG. 5A, device 500 can be configured in different ways. For example, line 506 may wrap around patch 502. Fastener 504 may be combined with patch 502 so that the entirety of patch 502 can act as a fastener such as an adhesive, glue, needle, screw, tape, magnet, suture, or combinations of these configurations or other configurations. In some embodiments of the present disclosure, device 500 may be inserted into a region of the heart. For example, patch 502 and fastener 504 may act together as a needle or screw, as depicted in FIGS. 5A, 5B, and 5C, with line 506 wrapped around it, to be inserted into a region of the heart. In this example, atrial defibrillation therapy can be applied to the heart after device 500 has been inserted into the heart.

According to another embodiment of the present disclosure, line 506 may be rigid so that after patch 502 or fastener 504 has been resorbed, line 506 may retain its form, attached to the heart. For example, device 500 may be inserted into a region of the heart. After patch 502 or fastener 504 has been resorbed, line 506 may still retain its coil form and remain in the region of the heart to perform atrial defibrillation therapy or other types of therapies. Line 506 may subsequently be removed by pulling on the portion of line 506 that remains outside the patient's body. Line 506 may be configured in a way such that if line 506 remained rigid, the subsequent removal from the patient's body would still not be obstructed. For example, line 506 configured in a rigid coil form would still be small enough to be removed from the patient's chest through a small incision or orifice.

In some embodiments, as depicted in FIG. 5D, line 506 may include at least two parts. The first part is an electrically conductive wire 507 that extends along line 506. As shown in FIG. 5D, the wire 507 can extend into patch 502 in such a way as to limit or reduce exposure of the wire 507 to body fluids and tissue other than the target heart tissue. In some embodiments, line 506 can include a covering or coating 509 that insulates surrounding tissue from the wire 507. This coating 509 preferably extends along the wire 507 at least until the wire 507 extends into the patch 502. In some embodiments, the coating 509 will extend into the patch 502 over the wire 507, as shown in FIG. 5D, to account for resorption of the patch 502 and to maintain a protective covering over the wire 507 after implantation into the patient's body.

Some embodiments of the patch 502 and line 506 are depicted in FIGS. 5E, 5F, and 5G. In some embodiments, the patch 502 comprises solely the fastener 504, and the fastener 504 is applied directly to the heart tissue. In some embodiments, the line 506 can be configured such that it provides varying depths within the patch 502, as depicted in FIG. 5F. This configuration may provide select points for targeted treatment of the heart tissue while still permitting low charge density through other portions of the patch 502. In some embodiments, as depicted in FIG. 5G, the device can include a fastener 504 portion and a substrate 505 portion. In this configuration, the fastener 504 can function as a adhesive and the substrate 505 portion can function to provide stability to the device. In some embodiments, the line 506 can be configured to be positioned in both the substrate 505 and the fastener 504, as shown in FIG. 5G.

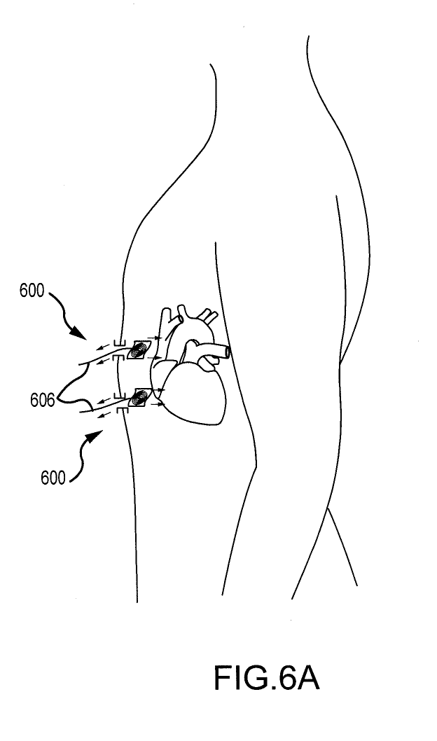

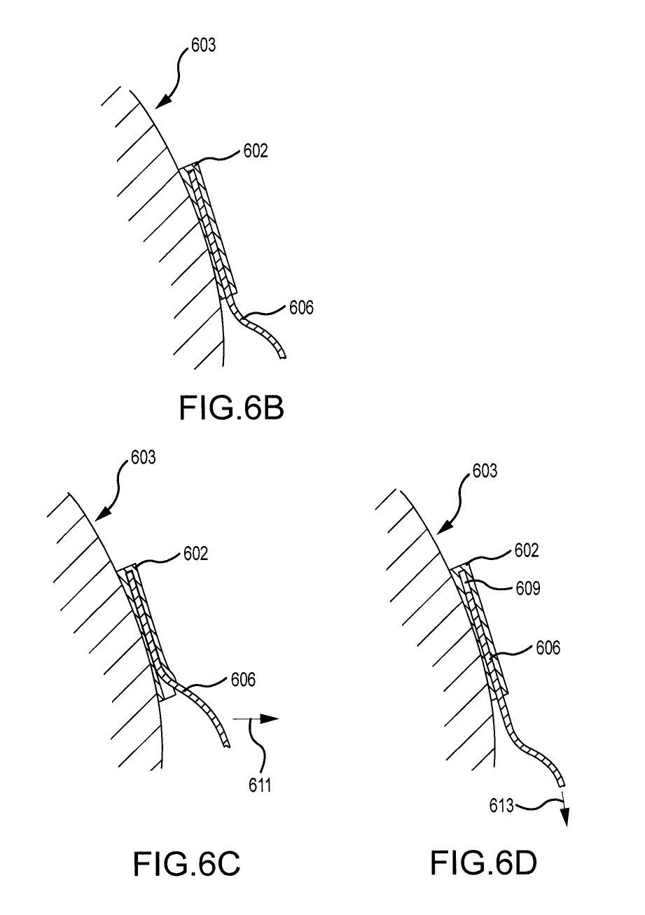

FIG. 6A is an illustration of implanting and removing the device, in accordance with some embodiments of the present disclosure.

Device 600 may be implanted and disposed into the body through laparoscopic, thoracoscopic, or other minimally invasive surgeries, in accordance with some embodiments of the present disclosure. Without the need for extensive surgeries, such as open chest surgery, device 600 may be implanted into the patient through small incisions made in the body. For example, a small incision can be made in the patient's chest so that device 600 can be inserted directly into the patient to be disposed in or on the patient's heart. Device 600 can be inserted into other parts of the body as well using the same technique.

In accordance with some embodiments of the present disclosure, a portion of device 600 preferably remains outside of the patient's body after device 600 has been implanted. Thus, the removal of device 600 would be relatively simple. For example, by withdrawing from the patient's body line 606, the device 600 can be removed from the patient's body through a small opening in the patient's skin. The removal of device 600 can be made easier by applying a gel or lubricant to device 600. As mentioned above, in some embodiments, the device 600 comprises bioresorbable components and nonbioresorbable components. In some embodiments, the device 600 is inserted into a patient through an incision in the patient's skin. The device 600 is attached to an atrial region of the patient's heart, with a distal portion of the line 606, at the heart, extending from the device 600 through the incision in the skin, and a proximal portion of the line 606 remaining outside of the patient's body. The incision is substantially closed, or is closed around line 606. If atrial fibrillation is detected, application of atrial defibrillation therapy is applied. After a monitoring period has passed, the line 606 is withdrawn proximally, during a nonsurgical procedure, from the patient. Accordingly, the disclosure provides methods, devices, and systems that provide for a device, having both bioresorbable and nonbioresorbable components, to be inserted into the body of a patient, for atrial fibrillation therapy, during a surgical procedure and, after a monitoring period, removal of the nonbioresorbable components of the device from the patient through a nonsurgical procedure.

FIGS. 6B, 6C, and 6D illustrate schematic cross-section views of some embodiments of the device. FIG. 6B depicts the device, having a patch 602 and a line 606 attached to body tissue 603. In some embodiments, the body tissue 603 can be epicardium, if the patch 602 is positioned under the pericardium, and in some embodiments, the body tissue 603 can be the pericardium. FIGS. 6C and 6D depicts different embodiments of how the line 606 is separated from the patch 602 upon removal of the device from the patient's body. In FIG. 6C, the line 606 is pulled in the direction of arrow 611. The force of the line 606 being pulled along arrow 611 will cause the line 606 to tear through the patch 602 and separate from the patch 602 while the patch 602 remains within the body. FIG. 6D depicts the line 606 being removed from the patch 602 by pulling the line 606 along the arrow 613, which causes the line 606 to slip out of the channel 609 that it resided in within the patch 602. In some embodiments, both methods depicted in FIGS. 6C and 6D are used to separate the line 606 from the patch 602 and to remove the line 606 from the patient's body.

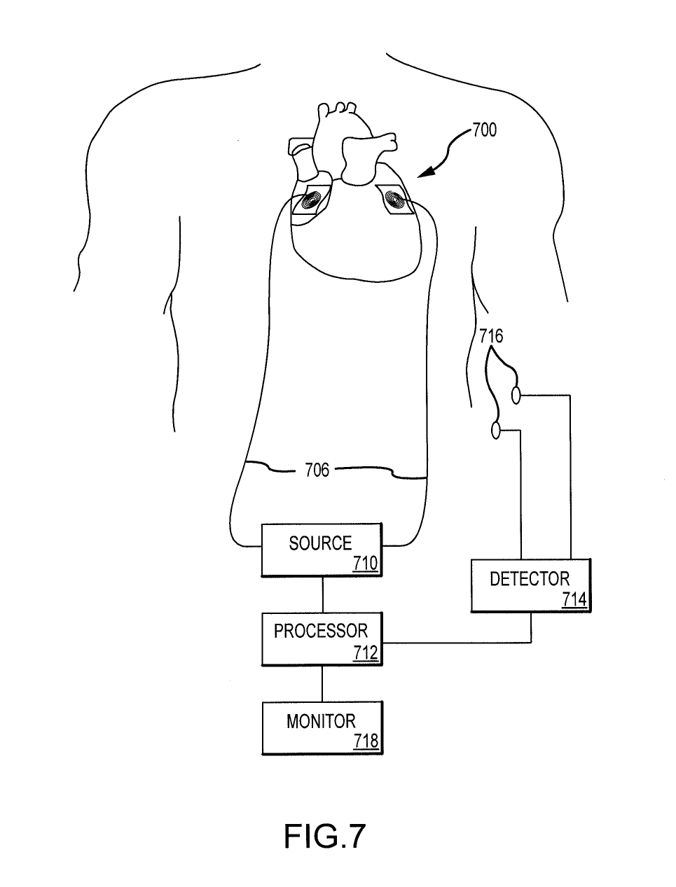

FIG. 7 illustrates the device with a control system, in accordance with some embodiments of the present disclosure. Device 700 can be controlled by the control system shown in FIG. 7. In accordance with some embodiments of the present disclosure, processor 712 is attached to line 706, source 710, detector 714, and monitor 718 while source 710 is also attached to line 706. Detector 714 is preferably attached to line 706 and monitor 718 is also attached to line 706.

In some embodiments, processor 712 controls the operation of device 700. For example, processor 712 may configure source 710 to provide power to line 706 as a pacing electrode. Source 710 may provide power from an outlet, batteries, or from a combination of both sources. Device 700 may be configured to provide atrial defibrillation therapy to the heart, which includes the application of electrical energy. Thus, source 710 may provide energy to the pacing electrodes for the treatment of atrial fibrillation. In other embodiments, source 710 may provide more or less electrical energy depending on different types of treatments for other cardiac arrhythmia. In another embodiment of the present disclosure, device 700 may be configured to provide atrial defibrillation therapy to the heart using drug therapy. For example, source 710 may store drugs and/or additives. Source 710 can then be configured to feed various drugs or additives into the heart by using line 706 as a tube or catheter. In one exemplary operation for treating atrial fibrillation, source 710 may feed drugs such as ibutilide directly into the heart by using line 706 as a tube or catheter for delivery.

Processor 712 may also configure detector 714 to monitor heart activity. Sensor 716 may be placed externally or internally to provide patient information to detector 714. For example, sensors 716 and detector 714 may monitor heart activity for heart rate, atrial fibrillation, ventricular fibrillation, or other cardiac arrhythmia. Processor 712 may configure device 700 to apply atrial defibrillation therapy upon detection of atrial fibrillation. Device 700 may also be configured to apply different types of therapies for different types of treatments. Monitor 718 may be configured to display information related to the patient, such as heart activity, power levels of the electrode, drug information, patient's information, or any other information relevant to the treatment. Although shown separately, source 710, processor 712, detector 714, sensors 716, and monitor 718 may be integrated as one unit without departing from the scope of the present disclosure.

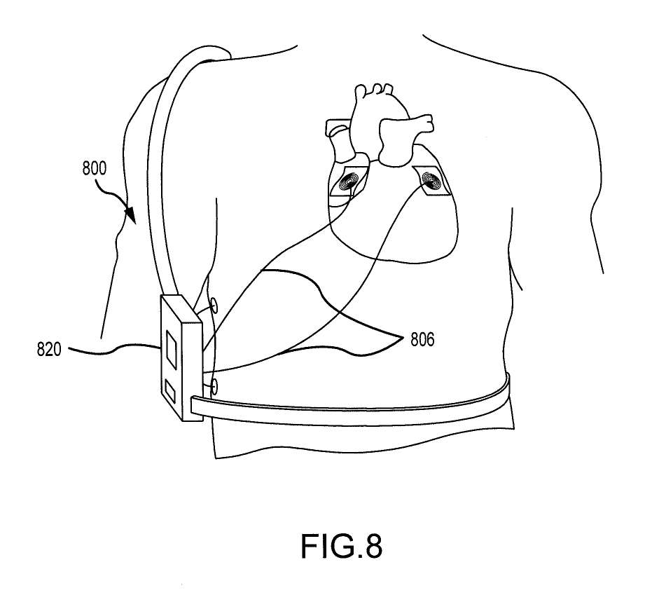

FIG. 8 illustrates an external configuration of the control system of the device, in accordance with some embodiments of the present disclosure.

Control 820 may incorporate a source, processor, detector, and monitor in accordance with some embodiments of the present disclosure. Control 820 can configure device 800 for different therapies related to the monitoring and treatment of the heart. For example, control 820 can monitor heart activity and configure device 800 to apply atrial defibrillation therapy to the heart upon detection of atrial fibrillation. Control 820 can supply the power for the electrodes or drugs to be administered directly to the heart. Control 820 may also include a monitor to show a user information related to the treatment.

In some embodiments of the present disclosure, control 820 can be battery powered and strapped onto the patient. This provides the benefit of mobility and portability for the patient, as well as cost savings because the patient will not have to stay in the hospital connected to larger control systems for the monitoring and treatment of the heart. In some embodiments of the present disclosure, device 800 can be configured to be waterproof. Thus, a patient may still be able to utilize device 800 even if device 800 gets wet, such as during a shower or in the rain. After treatment of the heart is concluded, device 800 may be removed from the patient by unstrapping control 820 and withdrawing line 806 from the patient's body.

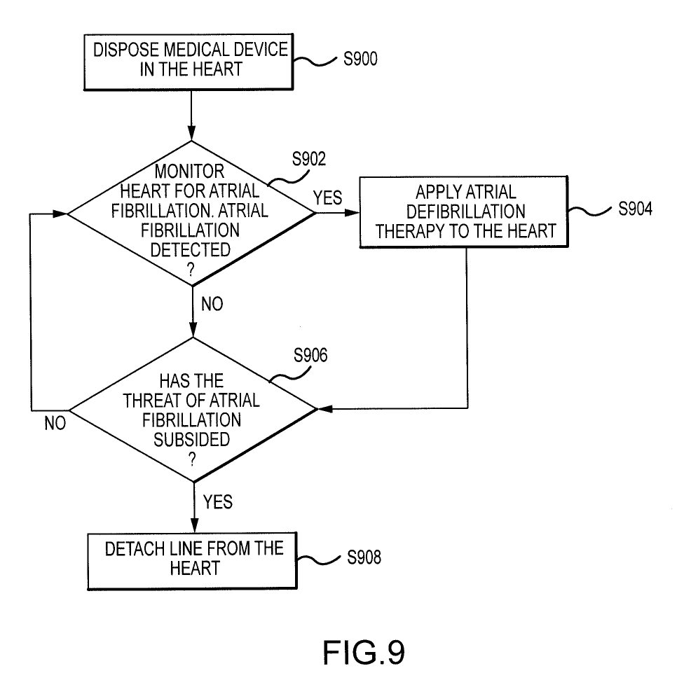

FIG. 9 illustrates a flowchart of a method for treating atrial fibrillation, in accordance with some embodiments of the present disclosure.

According to some embodiments of the present disclosure, S900 provides a method of disposing a device in or on the heart. For example, a device can be disposed in or on the heart during open heart surgery. The device can be attached to the heart utilizing different types of fasteners such as adhesives, glue, needles, screws, tape, magnets, suture, or combinations of these materials or other materials. At the conclusion of surgery, the chest of the patient can be closed up leaving a portion of the device outside of the patient's body. For example, the tail end of a wire, suture, electrode, catheter, string, cord, thread, rope, tube, or any combinations of these materials or other materials, can be left outside of the patient's body at the conclusion of surgery.

Another method of disposing a device in the heart involves utilizing laparoscopic, thoracoscopic, or any other minimally invasive surgery techniques. The device can be inserted through small incisions made in the patient to attach to the heart. Similarly, a portion of the device can be left outside the patient's body after the device has been disposed in the heart to allow for the easy removal of the device in the future after treatment of the heart has concluded.

In accordance with another embodiment of the present disclosure, S902 provides a method of monitoring the heart for atrial fibrillation. Internal sensors attached to the patch of the device, or external sensors, may be utilized to monitor heart activity such as the heart rate. The monitoring can be performed through external control systems that are used in hospitals or through portable, battery-operated control systems. Upon detection of abnormal heart activity, the processor of the control system may determine how to act in response. For example, the processor may direct the device to apply atrial defibrillation therapy to the heart upon detection of atrial fibrillation.

In accordance with some embodiments of the present disclosure, S904 provides a method of applying atrial defibrillation therapy to the heart upon detection of atrial fibrillation. Atrial defibrillation therapy may include providing drug therapy to the heart. For example, additives, such as drugs, can be added to the patch of the device, which would then be directly applied to the heart for treatment after the patch is attached to the heart. In some operations, an external control system can feed drugs such as ibutilide into a tube or catheter directly into the heart. In accordance with some embodiments of the present disclosure, atrial defibrillation therapy may include applying electrical energy to the heart. Electrodes may be used for this purpose and an external control system can provide the power needed for this therapy. For example, up to about 150 joules of energy can be provided to the electrodes to apply atrial defibrillation therapy to the heart.

If atrial fibrillation is not detected, then the device may continue to monitor the heart until the threat of atrial fibrillation has subsided. In accordance with some embodiments of the present disclosure, S906 provides a method of determining whether the threat of atrial fibrillation has subsided. After open heart surgery, many patients face the likelihood of developing temporary atrial fibrillation because of the heart's unstable state after surgery. Thus, patients may face up to many days with the possibility of temporary atrial fibrillation. Consequently, the threat of atrial fibrillation may subside considerably after this window of time has passed. Alternatively, a doctor may determine that the patient is not threatened with atrial fibrillation anymore. If this is the case, the device may then be removed.

In accordance with some embodiments of the present disclosure, S908 provides a method of detaching a line from the heart. Because at least a portion of the device is left outside of the patient after disposing the device in the heart, detaching the line from the heart is relatively simple. The line may be detached from the patient's heart by pulling on the tail end of the line, which may include the tail end of a wire, suture, electrode, catheter, string, cord, thread, rope, tube, or any combinations of these materials. Lubrication can be applied to the line to reduce friction and resistance as it is being pulled out.

In some embodiments of the present disclosure, the rest of the device will be or will have already been resorbed by the body. Thus, there is no need to physically remove the patch or fastener. In some embodiments, the patch or fastener may not be bioresorbable or biodegradable and detaching the line from the heart may also remove the patch and fastener from the body as well. In such cases, patch and fastener may be configured to be small enough in shape to fit through the small incision made in the patient. In some embodiments, invasive surgery, such as open heart surgery, may be performed to completely remove the device.

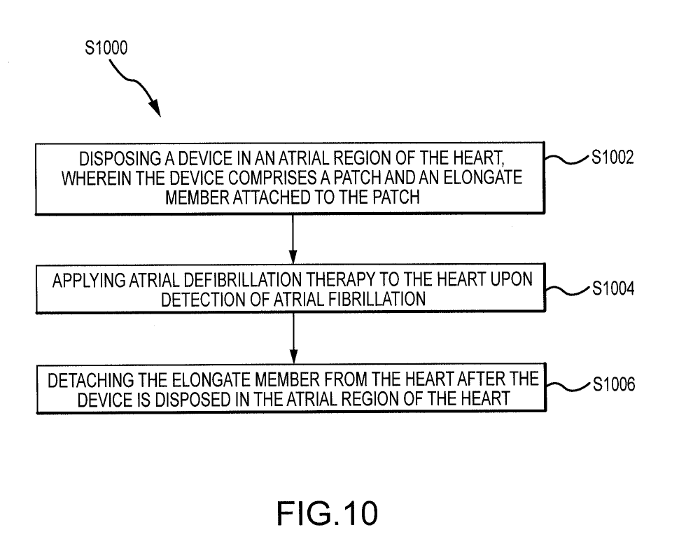

FIG. 10 illustrates a method S1000 for treating temporary atrial fibrillation, in accordance with various embodiments of the disclosure. Method S1000 may comprise the steps of: disposing a device in an atrial region of the heart, wherein the device comprises a patch and an elongate member attached to the patch (S1002); applying atrial defibrillation therapy to the heart upon detection of atrial fibrillation (S1004); and detaching the elongate member from the heart after the device is disposed in the atrial region of the heart (S1006). According to some embodiments, method S1000 comprises the step of detecting atrial fibrillation.

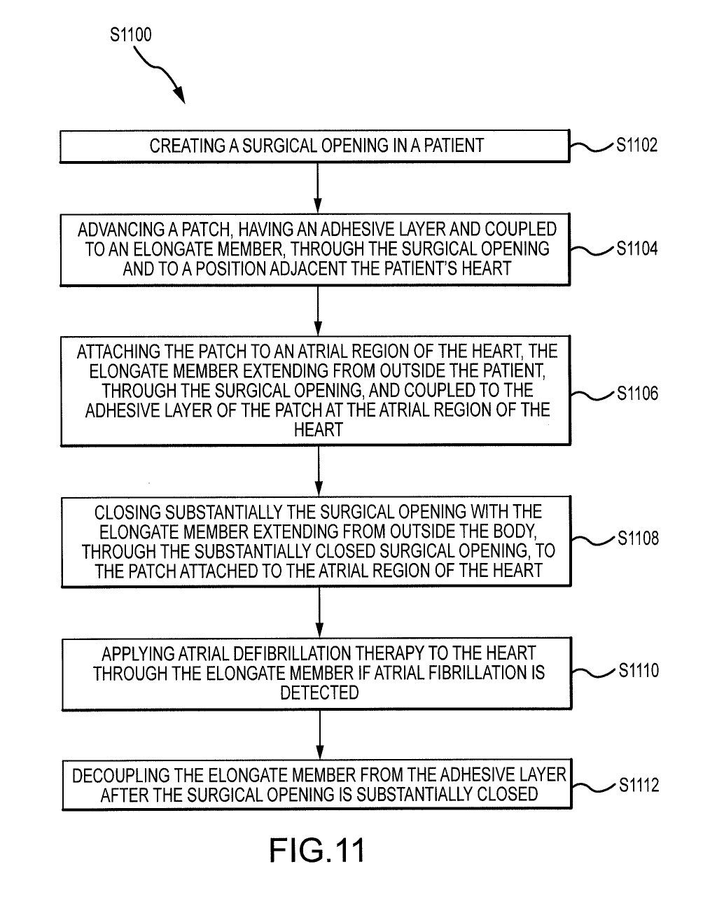

FIG. 11 illustrates a method S1100 of treating atrial fibrillation, in accordance with various embodiments of the disclosure. Method S1100 may comprise: creating a surgical opening in a patient (S1102); advancing a patch, having an adhesive layer and coupled to an elongate member, through the surgical opening and to a position adjacent the patient's heart (S1104); attaching the patch to an atrial region of the heart, the elongate member extending from outside the patient, through the surgical opening, and coupled to the adhesive layer of the patch at the atrial region of the heart (S1106); closing substantially the surgical opening with the elongate member extending from outside the body, through the substantially closed surgical opening, to the patch attached to the atrial region of the heart (S1108); applying atrial defibrillation therapy to the heart through the elongate member if atrial fibrillation is detected (S1110); and decoupling the elongate member from the adhesive layer after the surgical opening is substantially closed (S1112).

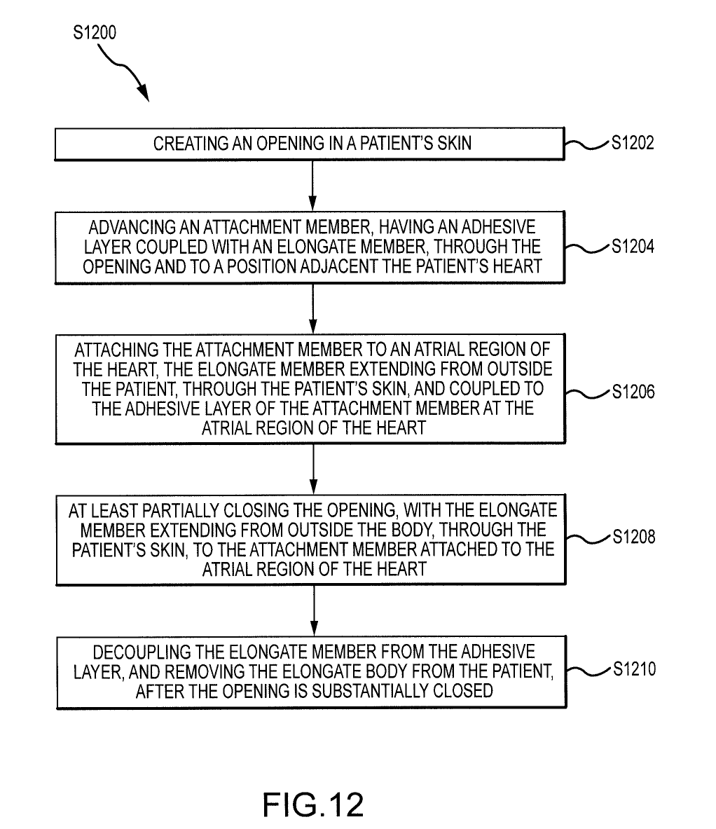

FIG. 12 illustrates a method S1200 of treating atrial fibrillation, in accordance with various embodiments of the disclosure. Method S1200 may comprise: creating an opening in a patient's skin (S1202); advancing an attachment member, having an adhesive layer coupled with an elongate member, through the opening and to a position adjacent the patient's heart (S1204); attaching the attachment member to an atrial region of the heart, the elongate member extending from outside the patient, through the patient's skin, and coupled to the adhesive layer of the attachment member at the atrial region of the heart (S1206); at least partially closing the opening, with the elongate member extending from outside the body, through the patient's skin, to the attachment member attached to the atrial region of the heart (S1208); and decoupling the elongate member from the adhesive layer, and removing the elongate body from the patient, after the opening is substantially closed (S1210).

The foregoing description of the preferred embodiment of the disclosure has been presented for the purposes of illustration and description. It is not intended to be exhaustive or to limit the disclosure to the precise form disclosed. Many modifications and variations are possible in light of the above teaching. It is intended that the scope of the disclosure not be limited by this detailed description.

There may be many other ways to implement the invention. Various functions and elements described herein may be partitioned differently from those shown without departing from the scope of the invention. Various modifications to these configurations will be readily apparent to those skilled in the art, and generic principles defined herein may be applied to other configurations. Thus, many changes and modifications may be made to the invention, by one having ordinary skill in the art, without departing from the scope of the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

What is claimed is:

1. A device, for treating postoperative atrial fibrillation, comprising:
an attachment member configured to attach to an atrial region of a patient's heart, the attachment member having an adhesion portion; and
an elongate member coupled to the attachment member and configured to extend from outside the patient, through the patient's skin, and to the attachment member when the adhesion portion is attached to the atrial region of the heart;
wherein the elongate member is configured to deliver electrical energy to the heart, sufficient to treat atrial fibrillation, upon detection of atrial fibrillation after the attachment member is attached to the atrial region of the heart; and
wherein the elongate member is configured such that, after the attachment member is attached to the atrial region, the elongate member is decouplable from the attachment member and is removable from the atrial region by withdrawing the elongate member through the patient's skin.

2. The device of claim 1, wherein the elongate member extends into the adhesion portion of the attachment member.

3. The device of claim 1, wherein the attachment member is bioresorbable.

4. The device of claim 3, wherein the attachment member is configured to attach to the atrial region of the heart with a bioresorbable glue.

5. The device of claim 4, wherein the bioresorbable glue is conductive of electricity.

6. The device of claim 1, wherein the electrical energy is up to about 150 joules.

7. The device of claim 1, wherein the elongate member comprises an epicardial electrode.

8. The device of claim 7, wherein the epicardial electrode is configured in a coil, rectangular, triangular, polygonal, circular, star, oval, accordion, or irregular line shape.

9. The device of claim 1, wherein detection of atrial fibrillation is performed through sensors internal to the patient.

10. The device of claim 1, wherein the attachment member is configured to remain attached to the atrial region of the heart during a monitoring period, which comprises a time period for a risk of atrial fibrillation to decrease at least more than 50% from an initial risk of atrial fibrillation following implantation of the device.

11. The device of claim 1, wherein the adhesion portion of the attachment member is configured to attach the device to the atrial region of the heart with an adhesive force that is greater than the coupling between the attachment member and the elongate member.

12. A method, of treating atrial fibrillation, comprising:
creating an opening in a patient's skin;
advancing an attachment member, having an adhesive layer coupled with an elongate member, through the opening and to a position adjacent the patient's heart;
attaching the attachment member to an atrial region of the heart, the elongate member extending from outside the patient, through the patient's skin, and coupled to the adhesive layer of the attachment member at the atrial region of the heart;
at least partially closing the opening, with the elongate member extending from outside the body, through the patient's skin, to the attachment member attached to the atrial region of the heart; and
decoupling the elongate member from the adhesive layer, and removing the elongate body from the patient, after the opening is substantially closed.

13. The method of claim 12, further comprising applying atrial defibrillation therapy to the heart through the elongate member to treat atrial fibrillation.

14. The method of claim 12, further comprising removing the elongate member from the patient without reopening the opening.

15. The method of claim 12, wherein at least partially closing the opening comprises closing the opening around the elongate member.

16. The method of claim 12, wherein attaching the attachment member comprises attaching the attachment member to the atrial region with bioresorbable glue.

17. The method of claim 13, wherein applying atrial defibrillation therapy comprises applying electrical energy.

18. The method of claim 17, wherein applying electrical energy comprises applying up to about 150 joules of electrical energy.

19. The method of claim 12, further comprising detecting atrial fibrillation.

20. A system, for treating atrial fibrillation, comprising:
an attachment member configured to attach to an atrial region of a patient's heart, the attachment member being sized and configured to be inserted into the patient through an open incision in the patient's skin;
an elongate member having a distal portion coupled to the attachment member and a proximal portion configured to extend outside the patient through the patient's skin when the attachment member is attached to the atrial region of the heart, the distal portion being configured to be decoupled from the attachment member and is removable from the atrial region by withdrawing the elongate member through the patient's skin after the incision in the patient's skin is closed;
a detector configured to detect atrial fibrillation of the patient's heart; and
a processor, in communication with the detector, configured to direct the application of atrial defibrillation therapy through the elongate member upon detection of atrial fibrillation by the detector.
